(19) United States
(12) Patent Application Publication
(10) Pub. No.: US 2010/0229878 A1
(43) Pub. Date: Sep. 16, 2010

(54) GARMENT TO PREVENT PRESSURE SORES

(76) Inventor: Stan D. Dill, Richmond, KY (US)

Correspondence Address: PASSE' INTELLECTUAL PROPERTY, LLC, 1717 BRASSFIELD RD., RALEIGH, NC, 27614, US

(21) Appl. No.: 12/402,028

(22) Filed: Mar. 11, 2009

(51) Int. Cl. A61F 5/30

(52) U.S. Cl. 128/891

(57) ABSTRACT

The present invention is a protective garment underpants having a first liner pad having a foam and polyester layers in the crotch to buttocks area and a second liner pad having an absorbent material and open cell foam layer useful for a male and a second garment for females and children where the absorbent layer is replaced by a pad similar to the first liner pad. The polyester layer and the absorbent material layer each have a directional nap running from the back of the garment forward.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates generally to padded protective undergarment for wearing alone or under pants and the like and, more specifically, to a padded protective undergarment for use by persons who must spend much of their time sitting or lying in one position, in a wheelchair, bed, or the like.

2. Description of Related Art

Persons who are confined to wheelchairs or beds for prolonged periods are subject to “bed sores” or, more accurately, “pressure sores”, which occur due to the breakdown of body tissues which are pressed or rubbed between bony protuberances of the body and the relatively unyielding surfaces upon which they sit or lay. Particularly this can be true where disease or injury has caused muscle atrophy and muscle tone is not present to provide normal levels of protection. Healthy strong muscles provide an environment, or “background”, against which the blood vessels push, and when this is gone, the blood vessels themselves atrophy, causing decreased blood supply to these tissues. This is one of the consequences of spinal cord or central nervous system disease or injuries, in addition to the loss of mobility.

Common sites of such sores are the groin and buttocks, caused by restricted circulation as well as compression of tissue between ilea, ischia or coccyx bones, and seats of wheelchairs or mattresses. Severe medical problems can result when one who is helpless and confined to a wheelchair or bed is left unattended for extended periods.

Persons with such immobility, including those who have lost the use of and, often the sensation in, their lower body, particularly their legs, are especially prone to suffer from this problem. Although pads for wheelchairs and pads for inclusion in clothing are available and effective for many purposes, and are widely used, they are not easily transferred from wheelchair to automobile, sofa, chair, or more commonly, to floor, for use when sitting thereon. Pad inserts for clothing are also not easily transferred between clothing.

It is estimated that more than two-thirds of pressure sores suffered by wheelchair-bound individuals occur during the time they are not in their wheelchairs or during times of heavy movement in the wheelchair. Partially, this is because it is difficult and awkward for such a person to move between another surface and a wheelchair, so they tend to stay in one place or the other. Also, however, it is inconvenient and sometimes ineffective to move a cushioning pad between wheelchair and floor, and to keep it in place when moving about on the floor.

These activities, combined with frequent sliding about, can cause pressure and abrasion sores and, sometimes, bruises or even lacerations from colliding with walls, corners, furniture, and other solid obstacles found everywhere in the average dwelling. These hazards are especially critical because of the lack of sensation in lower body parts which many persons have; even if they suffer an injury, they may be unaware of it until it has become infected. Minor collisions during normal daily activities, i.e., bumping into table legs, stubbing a toe, etc., can cause injuries which can deteriorate into serious medical problems if continually irritated by the same activities, and can have adverse health consequences. The skin of a person suffering from the kind of disabilities described herein can become so vulnerable to breakdown, that constant pressure or abrasion even by clothing seams can cause injury. Consequently, prevention becomes very important.

While the addition of padding to an existing garment eliminates some of the problems with other methods, the lack of sensation due to spinal cord injury makes the problem of knowing if an addition is correctly placed or has moved extremely dangerous. The fact that these very special needs still exist, in the face of the increasing number of persons having them, indicates that the shortcomings of the prior solutions are in great need of a better way to prevent pressure sores.

In U.S. Pat. No. 6,715,158 issued Apr. 6, 2004 to Hay, there is disclosed a padded device for placement on the outside of a garment to reduce pressure sores. The patent also discloses a number of other devices and methods used to reduce pressure sores.

### BRIEF SUMMARY OF THE INVENTION

The present invention relates to a new type of undergarment for use in preventing pressure sores for males, females and children. In particular, it has been discovered that an undergarment with a lined super absorbent wicking polyester or polyester blend shell and further has an absorbent crotch padding is an improvement on previously known methods and devices for the prevention of pressure sores. Other types of liners are preferable for men versus women and children as well as can be seen from the disclosure herein.

Accordingly, in one embodiment of the present invention there is disclosed a protective garment underpants for preventing or reducing the occurrence of pressure sores in an individual male comprising:
a) a stretchable polyester or polyester blend pant shell sized to fit at least between the waist and the thigh of the individual;
b) a first liner pad having an open cell foam pad inner layer and a wicking absorbent polyester or polyester blend fabric outer layer, the first liner pad positioned inside the shell from about the crotch area of the individual back to the buttocks of the individual;
c) a second liner pad comprising an open cell foam pad inner layer and a non-wicking absorbent material outer layer having a closed loop directional nap, the second liner pad positioned inside the shell from about the crotch area of the individual forward sufficient to essentially cover the genitals of the individual, the directional nap running away from the crotch area.

A protective garment underpants for preventing or reducing the occurrence of pressure sores in an individual adult female or a child comprising:
a) a stretchable polyester or polyester blend pant shell sized to fit at least between the waist and the thigh of the individual;
b) a first liner pad having an open cell foam pad inner layer and a wicking absorbent polyester or polyester blend fabric outer layer, the first liner pad positioned inside the shell from about the crotch area of the individual back to the buttocks of the individual; and
c) a second liner pad comprising an open cell foam pad inner layer and a wicking absorbent polyester or polyester blend fabric outer layer being of the same or different material as the first liner pad outer layer, the second liner pad positioned inside the shell from about the crotch area of the individual forward sufficient to cover the genitals of the individual, the second liner pad attached to the first liner pad along a common edge.

### BRIEF DESCRIPTION OF THE DRAWINGS

FIG. 1 is a perspective view of the front side of the male underpants with the underpants turned inside out.

FIG. 2 is a perspective view of the rear side of the male underpants with the underpants turned inside out.

FIG. 3a is a side perspective view of the second absorbent liner pad showing the directional nap.

FIG. 3b is a side view of the second absorbent liner with the embodiment where there are three layers.

FIG. 4 is a perspective view of the male present invention underpants turned right side out.

FIG. 5 is a perspective view of the front side of the female/child underpants with the underpants turned inside out.

FIG. 6 is a perspective view of the rear side of the female/child underpants with the underpants turned inside out.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the word “underpants” refers to a garment designed to be worn next to the skin in the crotch area of a man or woman and which can optionally be worn underneath outer garments such as pants. Pressure sores around the genital area are a common problem when sitting and the garment of the present invention is designed to prevent pressure sores in an individual in the areas prone to pressure sores covered by the garment underpants. By “crotch area” of the individual refers to the area of the pants that is positioned in the area where the legs meet the torso. This area is also called the genital area. The underpants can be briefs, shorts or full pants/tights or the like with the lining in the genital area being critical rather than how much of the body is covered.

As used herein “stretchable polyester or polyester blends” refers to any of numerous synthetic polyester polymers (and blends containing them) produced chiefly by reaction of dibasic acids with dihydric alcohols and used primarily as light, strong, weather-resistant textile fibers. The present invention polyester fiber is selected from any of the stretchable fabric polyesters or polyester elastomer type fabrics.

One of the embodiments of such stretchable polyester fabrics is spandex. Spandex is a polyester block copolymer of polyurethane and polyethylene glycol. Spandex consists of rigid and flexible segments in the polymer chain. The flexible segments give the fiber the stretch and the rigid segments hold the chain together. When force is applied, the folded flexible segments straighten out and then return to their original position when the force is removed. The proportions of flexible and rigid segments in the polymer chain determine the amount of stretch. Typically in the present invention the stretch should be sufficient to hold form against the skin so deniers of 500 and below are in some embodiments and below 100 in others.

Other polymers as noted can be mixed with the polyester fabric such as nylon, wool, acetate, rayon or the like to add durability or other desired properties to the polyester fabric. Such additional choices are within the skill in the art for manufacturing polyester blend fabrics.

As used herein “pant shell” refers to the polyester fabric garment itself which at a minimum can be sized to be placed between about the waist and thigh (similar to bicycle shorts or even smaller like low or midrise underpants or briefs) but can be any size such as shorts, full pair of pants, tights, body suit or the like. In the case of larger garments, the fabric for the shell beyond the minimum could be made with any fabric as long as the area around the genitals is the polyester fabric of the invention. The shell adds support and coverage in the area between the waist and thigh and holds the padding of the present invention. Since the fabric used is a stretch fabric, sizing of garments such as those shells made of spandex and the like are slightly smaller than the dimensions of an individual who will wear the garment.

The present invention has a different undergarment for males than for women or children. This is due to size and physical differences between the two. The under pants for a male have 2 separate liner pads inserted inside the shell, positioned against the skin. (See figures for a clear example of such positioning.) The “first liner pad” consists of an open cell foam pad inner layer (positioned against the inside of the shell). Open cell foam pads are readily available in a number of polymers and materials. One such material is foam esters. The thickness of the pad should be at least about an eighth of an inch up to about three-quarters of an inch or more but one in the art could determine the ideal thickness for such pad. The outer layer is a polyester fabric or polyester blend fabric that is both wicking and absorbent in nature. It is also an embodiment of the invention that the selected outer layer fabric is to have a non-directional nap. By “wicking” is meant the fabric has the ability to take water or moisture on its surface (from the wearer's skin touching the surface) and move it to a more internal location thus while being absorbent enough to hold water it can keep and hold it away from the skin or at least minimize such moisture contact. Examples of such materials are polyester suede, polyester velvet and short nap polyester fabric which all have a cut loop non-directional nap. These fabrics in general have a non-direction nap, usually a cut loop type. A fabric with a nap has raised or fuzzy fibers on the surface of the fabric. A “non-directional” nap refers to fibers that are in random directions or are essentially all upright. A “directional” nap refers to a fabric with nap fibers where the fibers all essentially lay in a particular direction. Resistance is minimal when rubbing the surface with the directional nap in the direction the fibers lay and increased when rubbing the surface of the fabric against the lay of the fibers.

The first liner pad is positioned inside the pant shell. One edge is positioned around the crotch area and the opposite edge is at the buttocks with the fabric itself covering both the genital crotch area and at least a small portion of the buttocks. The width of the first liner is about 5 to 12 inches but is sided to reach or even cover the buttocks area especially around the areas known to be prone to pressure sores in most individuals. It is cut and sewn together in pieces designed to fit the contour of the inside area of the pant shell. In one embodiment there are two pieces with a seam running in the middle as depicted in the drawings. A further understanding of the positioning and such of the first liner can be seen in the figures of the present invention.

The second liner pad is also at least a dual layer liner pad and comprises an open cell foam pad inner layer. This layer is similar to the inner layer of the first liner pad. The material and thickness of this layer can be the same or different than the first layer as desired. The outer layer is made of a non-wicking absorbent material having a directional nap. “Non-wicking absorbent” refers generally to absorbent materials which get evenly wet including on the surface. For the purpose of this invention it can have some wicking properties as long as it is significantly less wicking properties relative to the first liner pad wicking properties. Products such as terry cloth with a loop nap and the like are contemplated but any absorbent material with a similar directional nap is within the contemplation of the present invention. In one embodiment, the outer layer of the second line pad has a loop nap which is directional. In one embodiment of the present invention, the second liner pad has a third layer inner to the foam layer that can act as a liner for the cell foam acting to add properties such as stretch or water resistance to that part of the pad. Other features it might add are support since that area supports the male genitalia. In one embodiment, the third inner layer is a 40 denier tricot, a weave, or the like. One skilled in the art could easily determine the ideal liner based on the disclosure herein.

The second liner pad is positioned inside the pant shell. One edge is positioned around the crotch area and the opposite edge is positioned in the front part of the shell to cover the genital area. The width of the second liner is about 5 to 12 inches but is sided to cover the crotch forward area especially around the areas known to be prone to sweating such as the genitalia in most individuals. The second liner can be one or more pieces and cut and sewn or glued to fit the contour of the inside of the pant shell. The particular fabric has a directional nap and in the use in the present invention the direction nap faces away from the crotch of the individual (when looking at the piece in the crotch area). Accordingly, then the directional nap would be facing upward when looking at the second liner positioned over the front part of the shell. A further understanding of the positioning and such of the second liner can be seen in the figures of the present invention.

The first and second liner pads are joined together to form a continuous surface within the shell with no gaps and the like between them. That is true for both the male, female and children's versions. In all cases there will be separate first and second liner pads even when they are the same material.

In the making of the woman's or child's underpants there is a first and second liner, however, both the first and second liner are similar to the second liner for the male underpants in terms of layered construction. However, in one embodiment the materials are the same and in one embodiment different materials are used for the first and second liner. The exception is that the embodiment with the seam separating the first liner into left and right pieces is not as highly useful an embodiment for children and females as males. Because of anatomy differences, a female would most likely find the lack of seam from joint two pieces more comfortable while a male might find the seam comfortable. Likewise, the first and second liners are sewn together to form a single piece as depicted in the figures which follow.

The pieces of the garment underpants are joined together by any method and can be sewn, glued or the like by means known in the art for joining of fabrics either to form the shell, to layer the materials or the like. Edges are beveled where possible or desired and attached as flush as reasonably possible to prevent irritation from the joined parts of the invention. The exact size and shape of each liner pad is determined in a large part by the individual's size that the underpants will be worn by and by the location of the pressure points of the individual. One skilled in the art can adjust each pad and size the present invention accordingly without undue experimentation. In order to put the garment on, other devices or means could be added to the present invention undergarments such as handles or straps or the like. Velcro closures, zippers, ties, pockets flaps, buttons or any other means may be used to make it easier to don the garment or to urinate or use a catheter by providing a means for opening the garment in an appropriate manner to accomplish those acts. This may be especially useful for individuals with limited movement especially in the hands or for situations when the pants are being put on by a second individual, helper or assistant.

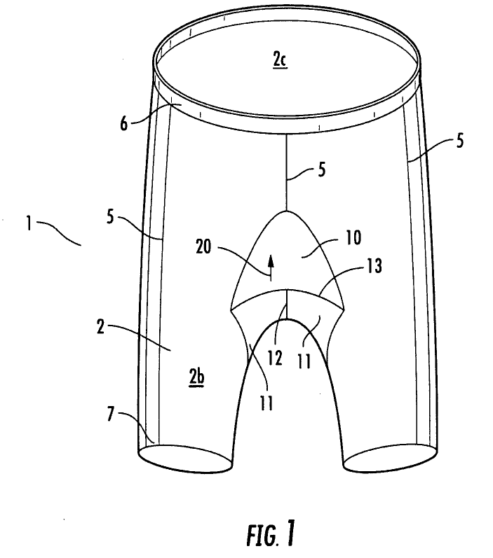

Now referring to the figures, FIG. 1 is an embodiment of the present invention protective garment underpants 1 turned inside out for the purposes of illustration. As can be seen in this view the pant shell 2, consists of several pieces of polyester fabric in this embodiment spandex sewn together at seams 5. Additionally, optional elastomer bands are sewn in at the waist 6 and leg cuffs 7 in order for these areas to hold the individual tightly. In this view the inside front 2b can be seen of underpants 2 and a portion of the outside rear 2c as well. In this view the direction of the directional nap 20 is indicated by an arrow.

The first liner pad 11 is shown in two pieces sewn together at seam 12 running from the crotch area towards the buttocks and attached to the pant shell 2. In this view it can be seen that the first liner 11 is positioned starting in the crotch. The second liner pad 10 is positioned in the inner front 2b of shell 2 connected to first liner pad 11 by seam 13.

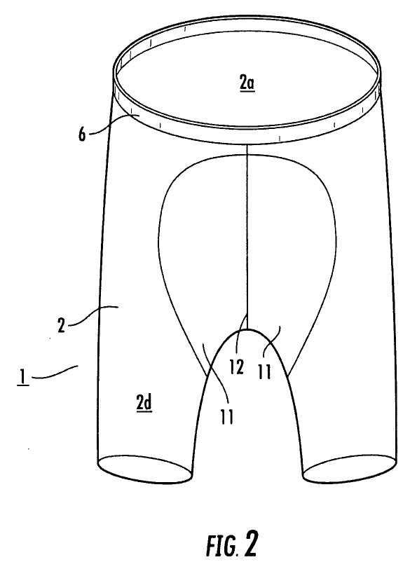

FIG. 2 shows the garment underpants 1 from the inside out rear facing the viewer showing inside back side 2d of shell 2 and a view of the outside front side 2a above. The remaining portion of the first liner pad 11 can be seen. The pad goes almost all the way up to the elastomeric waist 6 so that in use it essentially covers the entire buttocks area of the individual user in this embodiment.

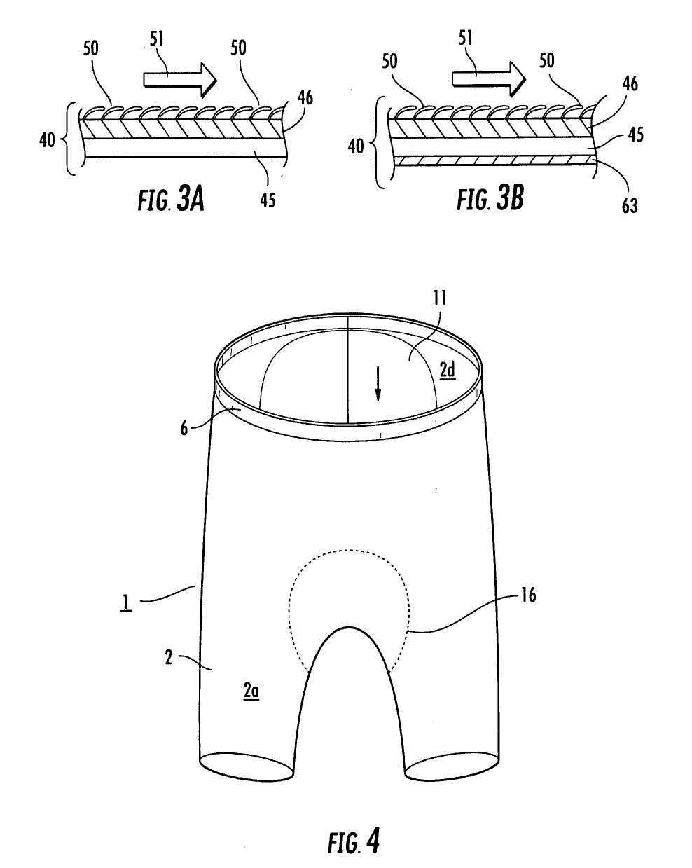

FIG. 3a shows a side view of a small piece of the second liner pad 40 for illustration purposes of directional nap (in this example a terry cloth outer layer). The open cell foam layer 45 is the part of the liner pad 40 that is mounted against the inside of the pant shell 2 from the previous figures. On top of that is the terrycloth fabric 46 having directional nap fibers 50. It is this layer fabric 46 that rests against the skin of the user. In this view, the two layers 45 and 46 are mated together and held in place by glue but any means of keeping them together such as melting or the like can be used within the present invention. Notice that all the directional fibers 50 are laying down in the same direction and the direction of the nap is indicated by nap arrow 51. This view helps indicate how the nap on the second liner pad is directionally oriented in the garment.

FIG. 3b shows an embodiment of the invention with the second liner pad 40 having a third layer 63 of material inner to open cell foam layer 45 and thus up against the shell 2. The third layer 63 is a stretchable liner material as described above. The outermost layer 46, such as terrycloth, shows the directional nap 50. In both 3a and 3b nap direction is indicated by arrow 51.

FIG. 4 is the present invention turned right side out for further illustration purposes. Front outside 2a is visible as is the top portion of inside rear 2d. None of the liner pads are visible except for a small portion of first liner 11 which can be seen in the little bit of the inside of the garment visible in this view on back inside 2d. The stitching 16 from second liner 10 can be seen from the front in the embodiment where the liner pads are sewn in place all the way through the garment shell 2. In this view the front side 2a is right side out and if the garment 1 were on an individual the individual would be facing the viewer. As noted above, while the particular figure depicts a pair of shorts, (of the size one would equate with bicycle shorts), other sizes involving tights or full body suits or the like could also be included in the present invention. One skilled in the art could easily change the size of the garment in view of the disclosure and figures herein.

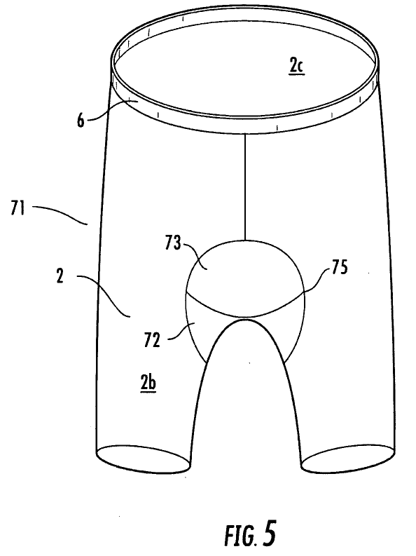

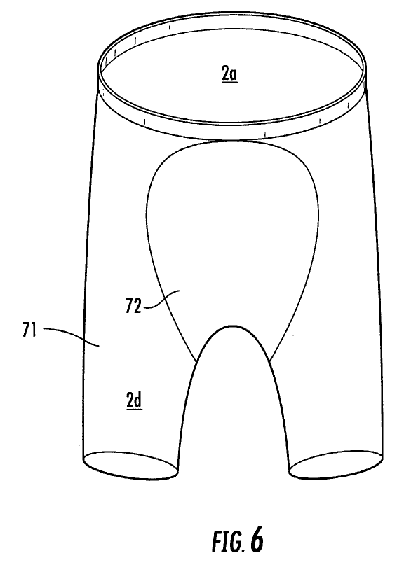

FIG. 5 shows the version of the underpants wherein the design is for a woman's or children's underpants 71. The shell for the woman's underpants 71 is identical to the male underpants having a first liner pad 72 and second liner pad 73. However, in this embodiment the non-wicking absorbent material 20 from FIG. 1 is replaced by more absorbent wicking polyester or polyester blend fabric. The fabric can be the same or different for each of the first and second liner pads. For example, the outer layer of one could be a napped polyester and the other a similar napped material. In other embodiments the first and second liner are the same wicking polyester fabric. One notes that there is always a seam between the two liners and seam 75 depicts the seam of attachment of the two liner pads. One additional difference in the child/woman's underpants though is the lack of seam 12 in the first liner. For this pair of pants a lack of seam would be more comfortable than having a seam and thus more wearable. One skilled in the art for all the pants would be able to cut and sew fabric to fit individual shells and noted elsewhere. One also notes that like the male counterpart pants, the first and second liner pads are sewn together to make a single liner piece. The exact placement of seams will take into account the difference in male and females (and children).

FIG. 6 shows the woman's/children's underpants 71 turned inside out but from the back side. First liner pad 72 is shown and comes up all the way to the waste to cover the buttocks entirely. This is not because of anatomical differences per se but can be determined for an individual based on the actual pressure point for that person such as from particular clothes, chairs and the like. Once again other features between the male and female garment are otherwise identical. One skilled in the art can clearly cut the particular pieces of material to fit in a given shape to create an underpants shell and create the layers described herein and sew them in place as directed herein.

One skilled in the art could easily substitute construction methods and means as well as substitute appropriate claims, fabrics, foams and the like within the scope of this invention. Nothing in the figures or the description is therefore intended to be limiting in scope for the claims which follow.

What is claimed is:

1. A protective garment underpants for preventing or reducing the occurrence of pressure sores in an individual adult male comprising:
a) a stretchable polyester or polyester blend pant shell sized to fit at least between the waist and the thigh of the individual;
b) a first liner pad having an open cell foam pad inner layer and a wicking absorbent polyester or polyester blend fabric outer layer, the first liner pad positioned inside the shell from about the crotch area of the individual back to the buttocks of the individual;
c) a second liner pad comprising an open cell foam pad inner layer and a non-wicking absorbent material outer layer having a closed loop directional nap, the second liner pad positioned inside the shell from about the crotch area of the individual forward sufficient to essentially cover the genitals of the individual, the directional nap running away from the crotch area.

2. A protective garment according to claim 1 wherein the first liner pad outer layer has a cut loop nap.

3. A protective garment according to claim 1 wherein the first liner pad comprises left and right pieces sewn together to form a single pad with a seam in the middle running from the crotch area toward the buttocks.

4. A protective garment according to claim 1 wherein the non-wicking absorbent material having a directional loop nap is terry cloth.

5. A protective garment according to claim 1 wherein the pant shell is a polyester elastomer.

6. A protective garment according to claim 5 wherein the polyester elastomer is spandex.

7. A protective garment according to claim 1 wherein the first liner pad outer layer is a wicking absorbent napped polyester fabric having a cut loop nap.

8. A protective garment according to claim 1 wherein there is an elastic waist.

9. A protective garment according to claim 1 wherein there is a means to open the garment in order for the individual to use the bathroom.

10. A protective garment according to claim 1 wherein the shell is shorts.

11. A protective garment according to claim 1 wherein the shell is a pair of pants.

12. A protective garment according to claim 1 wherein the second liner pad has a third innermost layer of a stretchable polyester fabric.

13. A protective garment underpants for preventing or reducing the occurrence of pressure sores in an individual adult female or a child of either sex comprising:
a) a stretchable polyester or polyester blend pant shell sized to fit at least between the waist and the thigh of the individual;
b) a first liner pad having an open cell foam pad inner layer and a wicking absorbent polyester or polyester blend fabric outer layer, the first liner pad positioned inside the shell from about the crotch area of the individual back to the buttocks of the individual; and
c) a second liner pad comprising and open cell foam pad inner layer and a wicking absorbent polyester or polyester blend fabric outer layer being of the same or different material as the first liner pad outer layer, the second liner pad positioned inside the shell from about the crotch area of the individual forward sufficient to cover the genitals of the individual, the second liner pad attached to the first liner pad along a common edge.

14. A protective garment according to claim 13 wherein the pant shell is a polyester elastomer.

15. A protective garment according to claim 14 wherein the polyester elastomer is spandex.

16. A protective garment according to claim 13 wherein the first liner pad and second liner pad outer layer are both a wicking napped polyester fabric with a cut loop nap.

17. A protective garment according to claim 13 wherein there is a means to open the garment in order for the individual to use the bathroom.

18. A protective garment according to claim 13 wherein the shell is shorts.

19. A protective garment according to claim 13 wherein the shell is a pair of pants.

20. A protective garment according to claim 13 wherein the polyester or polyester blend fabric has a cut loop nap.
